(19) United States
(12) Patent Application Publication
(10) Pub. No.: US 2024/0225666 A9
(43) Pub. Date: Jul. 11, 2024

(54) SYSTEM OF MODULAR REAMERS AND BROACHES

(71) Applicant: Zimmer, Inc., Warsaw, IN (US)

(72) Inventor: Katherine B. Lindsley, South Bend, IN (US)

(21) Appl. No.: 18/368,980

(22) Filed: Sep. 15, 2023

Related U.S. Application Data
(60) Provisional application No. 63/418,819, filed on Oct. 24, 2022.

(51) Int. Cl. A61B 17/16; A61B 17/14; A61B 17/88; A61F 2/46

(52) U.S. Cl. CPC A61B 17/1617; A61B 17/142; A61B 17/164; A61B 17/1662; A61B 17/88; A61F 2/4603; A61B 2017/0046

(57) ABSTRACT

A system of modular reamers and broaches can include a reamer and a broach. The reamer can be configured to ream a portion of the bone. The reamer can include a cutting edge and a handle. The cutting edge can extend longitudinally. The handle can extend longitudinally and can be removably attached to the cutting edge. The handle can be configured to rotate the reamer about a central axis of the reamer. The broach can be configured to prepare a medullary cavity of the bone to prepare the bone to receive one of the plurality of different implants. The broach can include a stem and a plurality of different broach heads. The stem can extend longitudinally. Each of the plurality of different broach heads can be configured to prepare the medullary cavity for one of the plurality of different implants.

### CLAIM OF PRIORITY

This application claims the benefit of U.S. Provisional Patent Application Ser. No. 63/418,819, filed on Oct. 24, 2022, the benefit of priority of which is claimed hereby, and which is incorporated by reference herein in its entirety.

### TECHNICAL FIELD

Embodiments described herein generally relate to reamers and broaches and, more specifically, to a system of modular reamers and broaches.

### BACKGROUND

Stem implants can be used to repair damaged hip and shoulder joints. There are multiple kinds of stem implants (e.g., fracture implants, revision implants, advanced revision implants, and limb salvage implants). Stem implants include a stem that can be implanted within a bone and an engagement mechanism for engaging with a second bone (or a second prosthetic component) installed within the second bone.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1 is a schematic diagram of a system of modular reamers and broaches, in accordance with at least one example of this disclosure.

FIG. 2 illustrates three configurations of a modular reamer, in accordance with at least one example of this disclosure.

FIG. 3 illustrates an example of a modular broach and an example of modular broach heads, in accordance with at least one example of this disclosure.

FIG. 4 is a schematic diagram of a modular reamer reaming a bone of a patient, in accordance with at least one example of this disclosure.

FIG. 5 is a schematic diagram of a modular broach preparing a bone of a patient for an implant, in accordance with at least one example of this disclosure.

FIG. 6 is a schematic diagram of an implant installed in a bone of a patient, in accordance with at least one example of this disclosure.

FIG. 7 illustrates a schematic view of a method, in accordance with at least one example of this disclosure.

### DETAILED DESCRIPTION

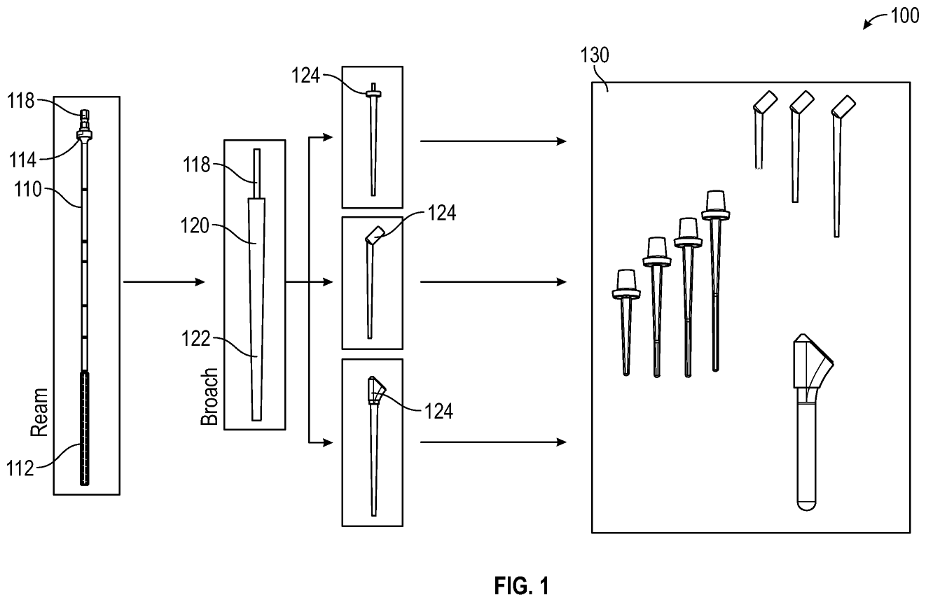

The inventors of the present disclosure have discovered that stemmed implants typically have their own instruments for the implantation process. Reamers and broaches are included in almost all surgical techniques to prepare a bone for these stems. However, each implant can have a different set of reamers and broaches depending on the specific length and geometry of the implant. The present disclosure can solve the need for different sets of reamers and broaches for each length and geometry of the implant. These instruments can be consolidated into a central set of instruments to seat all implants designed to include this type of workflow. The system having modular reamers and broaches will be discussed with reference to FIGS. 1-7 below.

FIG. 1 is a schematic diagram of a system of modular reamers and broaches (system 100). System 100 can be sent to a surgeon to prepare a bone of a patient to receive an implant. System 100 can work with a plurality of different implants. For example, the system 100 can prepare the bone of the patient to receive an advanced fracture implant, revision implant, and a limb salvage implant. In an example, the system 100 can include a plurality of reamers (reamers 110) and a plurality of broaches (broaches 120). The reamers 110 and the broaches 120 can be used to prepare a bone of the patient for a plurality of different implants (implants 130).

The reamers 110 can be configured to ream a portion of the bone. The reamers 110 can be modular to ream the bone of the patient in preparation for any of the implants 130. The reamers 110 can include different sizes. For example, the reamers 110 can include diameters different than the other reamers of the reamers 110. The different sizes of the reamers 110 can match various sizes of the implants 130. The reamer 110 can include a cutting edge 112 and a handle 114. The handle 114 can include a coupling mechanism 118.

The cutting edge 112 can be operable to ream the bone of the patient. The cutting edge 112 can extend longitudinally. The cutting edge 112 can be removably attached to the handle 114. The cutting edge 112 can have diameters to match the sizes of the implants 130. For example, the cutting edge 112 determines a diameter of the ream. Thus, the system 100 can have a plurality of different cutting edges 112 to change the diameter of the ream in the bone. In one example, the cutting edge 112 can attach directly to the handle 114.

The handle 114 can permit handling of the reamers 110 and provides a space between the cutting edge 112 and the coupling mechanism 118. The handle 114 can attach to a tool to apply a force to the handle 114 and rotate the reamers 110 about the central axis. The handle 114 can also extend longitudinally. The handle 114 can be removably attached to the cutting edge 112. In examples, the handle 114 can include labels or markings that indicate a size of the reamers 110 to help medical professionals pick the correct size of the reamers 110.

The coupling mechanism 118 can be engageable with a ratcheting t-handle, which can be used to rotate the reamers 110 about a central axis of the reamers 110. The handle 114 can extend longitudinally from the coupling mechanism 118. As the reamers 110 rotate about the central axis, the reamers 110, and more specifically, the cutting edge 112, can engage a bone of a patient to ream out the bone. In examples, the coupling mechanism 118 can be a quick-release adapter such that the reamers 110 can be quickly removed from the racheting t-handle, or any other device that can be used to rotate the reamers 110.

The broaches 120 can be inserted into a reamed bone of a patient to prepare a medullary cavity of the bone for any of the implants 130. The broaches 120 can be engageable by impacting the broach head into the bone so broach teeth can remove a part of the bone. The broach 120 can include a coupling mechanism 118, a stem 122, and a plurality of different broach heads 124.

The coupling mechanism 118 can attach any of the different broach heads 124 to any of the broaches 120. In examples, the coupling mechanism 118 can be engageable with the tool to advance the broaches 120. In examples, the coupling mechanism 118 can extend through the different broach heads 124. The coupling mechanism 118 can be a quick-release adapter such that the broaches 120 can be quickly removed from the broach heads 124. The coupling mechanism 118 can be integral with the stem 122. In an example, the connection mechanism 118 can be threaded, a morse taper, or any other connection type that can withstand the forces of the broaches 120.

The stem 122 can extend longitudinally from the coupling mechanism 118 such that the stem 122 can be inserted into the reamed bone of the patient. The system 100 can include various different sizes of the stem 122. For example, the different sizes of the stem 122 can each have a different diameter from the other of the stem 122. The stem 122 can taper radially towards a central axis as the stem 122 extends longitudinally away from a portion of the stem 122 that attaches to the plurality of different broach heads 124. The taper of the stem 122 can taper the bone of the patient to align with a stem of one of the implants 130. The alignment between the bone of the patient and the stem of one of the implants 130 can help support the implants 130 after installation in the bone of the patient.

The different broach heads 124 can be configured to fit onto the coupling mechanism 118 of the broaches 120 to prepare the medullary cavity of the patient for one of the implants 130. In examples, each of the different broach heads 124 can be configured to prepare the medullary cavity of the patient for a different kind of implant. For example, the different broach heads 124 can be configured to prepare the bone of the patient for a limb salvage implant, a revision stem implant, or an advanced fracture implant. The broaches 120 can receive each of the different broach heads 124 such that the different broach heads 124 are removably coupled to the broaches 120.

In an example, the system 100 can also include the implants 130. In another example, the surgeon can separately purchase and or procure the implants. For example, the surgeon can order the implants as they are used and can have the system 100 to prepare each patient for the implantation of any of the implants 130.

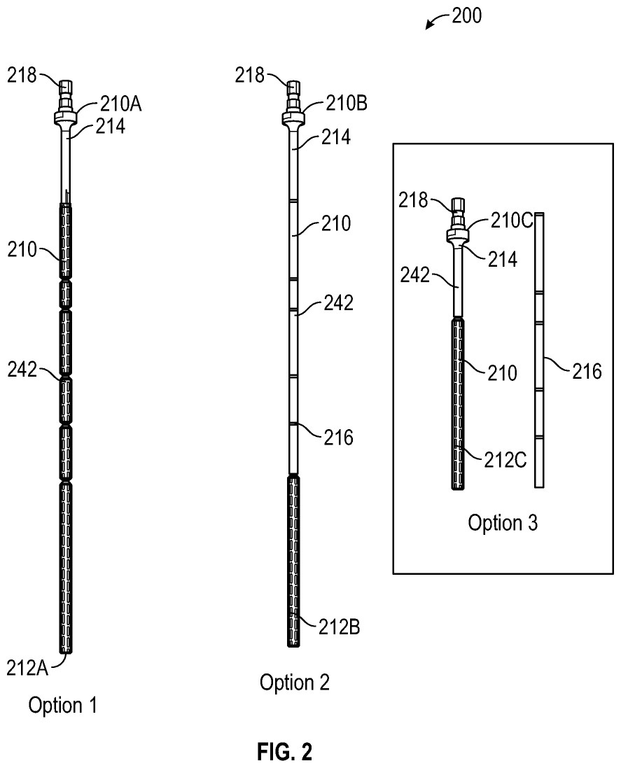

FIG. 2 illustrates an example of a modular reamer 210A, modular reamer 210B, and modular reamer 210C.

Each of the reamers 210 (e.g., the modular reamer 210A, the modular reamer 210B, the modular reamer 210C) can include a cutting edge 212 (e.g., a cutting edge 212A, a cutting edge 212B, a cutting edge 212C) to engage with a bone of the patient. In each of the reamers 110, the cutting edge 212 can be different lengths, diameters, or cutting edges. For example, the different cutting edges can be different cutting types (e.g., blades, grit, diamonds, any other surface that can be used to cut a bone of a patient, or the like).

The cutting edge 212A can extend longitudinally from a coupling mechanism 118. The cutting edge 212A can include a plurality of markings 242 thereon that can indicate a reaming depth of the modular reamer 210A. In examples, the markings 242 can be labeled with an alphanumeric reference such as to display a reaming depth to a surgeon during an operation. The cutting edge 212A can be removable from the reamers 210 such that the cutting edge 212A can be replaced as the cutting edge 212A becomes worn. In an operable example, the extended longitudinal length of the cutting edge 212A can improve the efficiency of reaming while reaming a long passage within the bone.

The modular reamer 210B can include an extension 216. The extension 216 can increase the reaming depth of the reamer 210B. The extension 216 can be removably attached between the cutting edge 212B and the handle 214. The system 100 can include a plurality of the extension 216, where each of the extension 216 can have a different length than the others. In examples, the extension 216 can include a plurality of markings (markings 242). The markings 242 can be formed, etched, marked, or the like, on the extension 216. The plurality of markings 242 can indicate a cutting depth of the reamer 210B. In examples, the markings 242 can be labeled with an alphanumeric reference such as to display a reaming depth to a surgeon during an operation. In an operable example, the extension 216 can enable the modular reamer 210B to ream out deep passages within the bone.

The modular reamer 210C can have the extension 216 removed such that the cutting edge is directly coupled to a handle 214. The handle 214 can include a plurality of markings (markings 242). The markings 242 can be formed, etched, or the like, on the handle 214. The plurality of markings 242 can indicate a cutting depth of the reamer 210C. In examples, the markings 242 can be labeled with an alphanumeric reference such as to display a reaming depth to a surgeon during an operation. In an operable example, the modular reamer 210C can be an efficient reamer for short passages. Moreover, the length of the modular reamer 210C can reduce the cost of modular reamer 210C because less material is used to make modular reamer 210C.

Each of the reamers 210 can include a coupling mechanism 218. The coupling mechanism 218 can make the reamers 210 engageable with a tool such that the tool can rotate the reamers 210 about a central axis of the reamers 210.

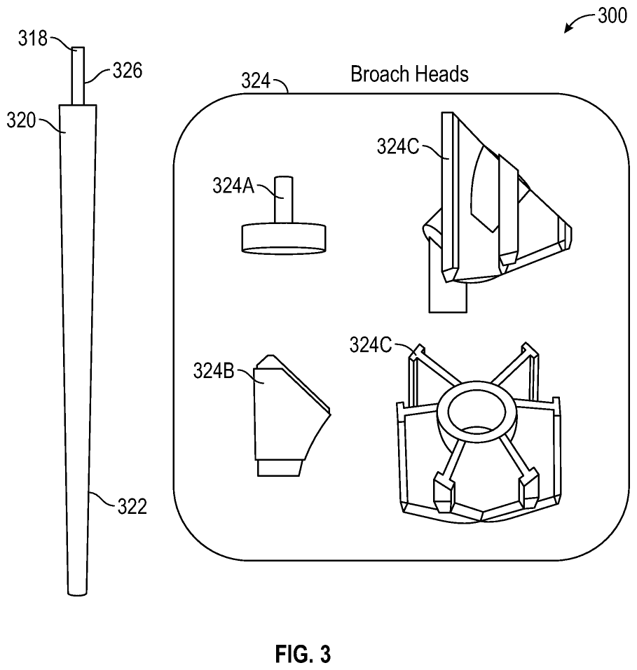

FIG. 3 illustrates an example of a system 300 including one or more modular broaches 320 and an example of a plurality of different broach heads (different broach heads 324).

The modular broach 320 can be inserted into a reamed bone of a patient to prepare a medullary cavity of the bone for any of the implants. The modular broach 320 can include a stem 322. The stem 322 can extend longitudinally from a coupling mechanism 318. An attachment portion 326 can be formed in the coupling mechanism 318. The attachment portion 326 can receive any of the different broach heads 324. Together the stem 322 and the different broach heads 324 can prepare a bone to receive an implant.

The system 300 can include a broach head 324A. The broach head 324A can be a limb salvage broach. Thus, the broach head 324A can prepare a reamed bone for implantation of a limb salvage implant. The broach head 324A can be removably attached to a tool via the coupling mechanism 318 to advance the stem 322 and the broach head 324A.

The system 300 can include a broach head 324B. The broach head 324B can be a revision broach. Thus, the broach head 324B can be used to prepare a reamed bone to implant an identity long revision broach. The broach head 324B can be removably attached to a tool to advance the stem 322 and the broach head 324B.

The system 300 can include a broach head 324C. The broach head 324C can be a fracture and revision broach head. Thus, the broach head 324C can be used to prepare a reamed bone for implantation of a fracture and revision implant. The broach head 324C can be removably attached to a tool to advance the stem 322 and the broach head 324C.

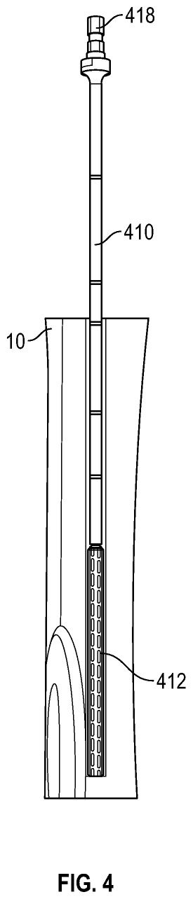

FIG. 4 is a schematic diagram of a modular reamer (reamer 410) reaming a bone 10. The reamer 410 can include a cutting edge 412 and an coupling mechanism 418. In an operable example, the coupling mechanism 418 can attach to a tool that rotates the reamer 410 about a central axis such that the cutting edge 412 engages the bone 10. As shown in the figure, a surface of the bone 10 can be resected to prepare the bone 10 to receive the reamer 410. The reamer 410 can ream the bone 10 with axial force along a longitudinal axis of the bone 10. The reamer 410 reams the bone 10 to prepare the bone 10 to receive a broach. In examples, one or more of the reamer 410 can be used to alter a depth reamed in the bone 10.

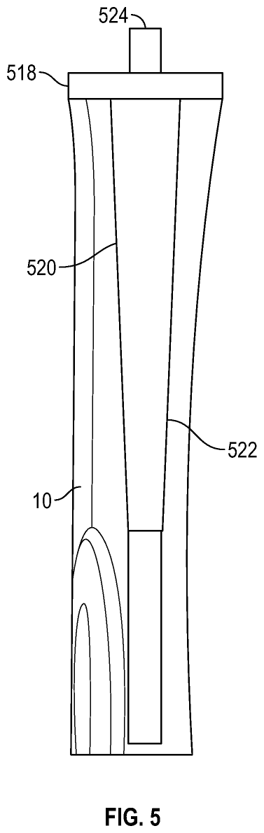

FIG. 5 is a schematic diagram of a modular broach (broach 520) preparing the bone 10. As shown in FIG. 5, the broach 520 can include a stem 522 and one of the plurality of different broach heads (different broach heads 524). The stem 522 can taper radially inward as the stem 522 extends away from a coupling mechanism 518 on the stem 522. The coupling mechanism 518 can be engageable with the different broach heads 524 to removably couple the different broach heads 524 to the stem 522. In an operable example, the broach 520 can be inserted into a portion of the bone 10 reamed by the reamer 410. A tool, attached to the coupling mechanism 518, can be attached to the broach 520 to advance the broach 520 and chip away the bone 10. As discussed above, the broach 520 can use a different kind of the different broach heads 524 to prepare the bone 10 for a different type of an implant.

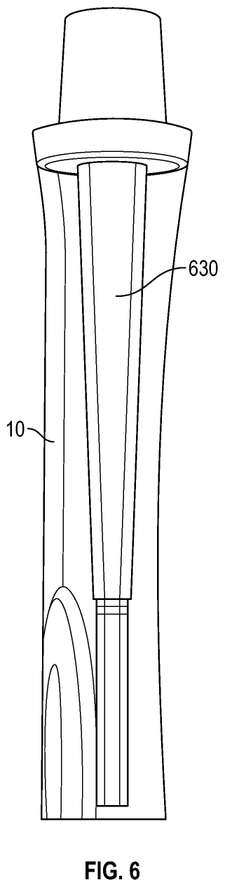

FIG. 6 is a schematic diagram of an implant 630 installed in the bone 10. In the example of FIG. 6, the implant 630 can be an advanced fracture implant. In another example, the implant 630 can be a limb salvage implant, a revision implant, or the like. In an operable example, a system (e.g., the system 100, the system 200, or the system 300) can be used to prepare the bone 10 for each of the different kinds of implants 630 by using an appropriate reamer and broach to prepare the bone 10 for the implant 630.

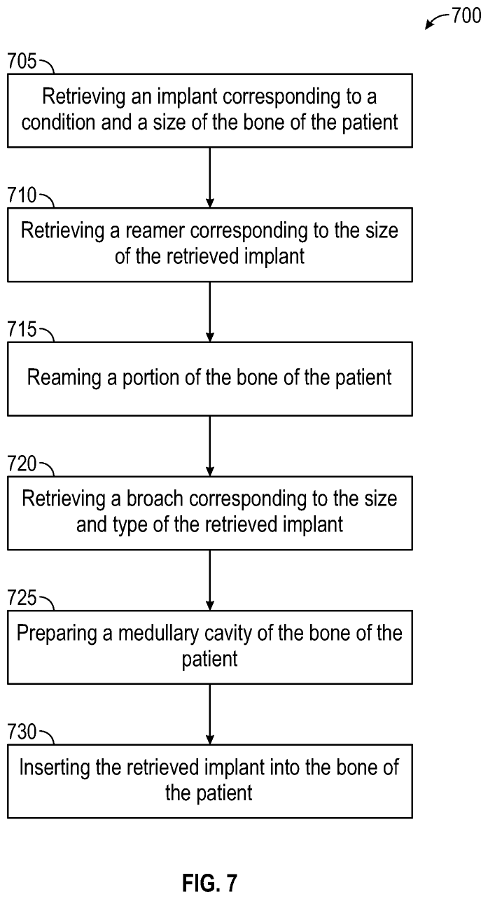

FIG. 7 illustrates a schematic view of a method 700, in accordance with at least one example of this disclosure. The method 700 can be a method of system of modular reamers and broaches. More specific examples of the method 700 are discussed below. The steps or operations of the method 700 are illustrated in a particular order for convenience and clarity; many of the discussed operations can be performed in a different sequence or in parallel without materially impacting other operations. The method 700 as discussed can include operations performed by multiple actors, devices, or systems. It is understood that subsets of the operations discussed in the method 700 can be attributable to a single actor, device, or system that could be considered a separate standalone process or method.

At operation 705, the method 700 can include retrieving an implant corresponding to a condition and the size of the bone of the patient. For example, a surgeon, nurse, medical technician, or the like can retrieve an implant to heal the ailment of a patient. The medical professional can retrieve a limb salvage implant, a 1 revision implant, or a fracture implant depending on the needs of the injuries of the patient. Moreover, the size of the implants can be selected based on the size or diameter of the bone of the patient.

At operation 710, the method 700 can include retrieving a reamer corresponding to the size of the retrieved implant. The medical staff can grab a reamer of an appropriate size to match the implant selected. For example, the size or diameter of the reamer can equal or be slightly less than the implant.

At operation 715, the method 700 can include reaming a portion of the bone of the patient. As discussed with reference to FIG. 4, the reamer can ream the bone to prepare the bone for the broach.

At operation 720, the method 700 can include retrieving a broach corresponding to the size and type of the retrieved implant. The broach can be selected based on the type of implant retrieved by the medical professional. Moreover, the broach size can match the size of the implant selected by the medical professional.

At operation 725, the method 700 can include preparing a medullary cavity of the bone of the patient. The broach can be used to machine away the bone. For example, the broach teeth advance to chip and wear the bone of the medullary cavity to prepare the bone for the implant.

At operation 730, the method 700 can include inserting the retrieved implant into the bone of the patient. In examples, the implant can be inserted into the prepared portion of the bone.

### Additional Notes & Examples

Example 1 is a system of modular reamers and broaches configured to prepare a bone of a patient for one of a plurality of different implants, the system comprising: a reamer configured to ream a portion of the bone, the reamer including: a cutting edge extending longitudinally; and a handle extending longitudinally and removably attached to the cutting edge, the handle configured to rotate the reamer about a central axis of the reamer; and a broach configured to prepare a medullary cavity of the bone to prepare the bone to receive one of the plurality of different implants, the broach including: a stem extending longitudinally; and a plurality of different broach heads, each of the plurality of different broach heads configured to prepare the medullary cavity for one of the plurality of different implants.

In Example 2, the subject matter of Example 1 optionally includes wherein the cutting edge of the reamer comprises a plurality of indentations, each of the plurality of indentations indicative of a cutting depth of the reamer.

In Example 3, the subject matter of any one or more of Examples 1-2 optionally include wherein the handle of the reamer comprises a coupling mechanism, the coupling mechanism engageable with a tool to rotate the reamer about the central axis, and wherein the handle of the reamer also comprises a plurality of markings thereon, each of the plurality of markings indicative of a cutting depth of the reamer.

In Example 4, the subject matter of any one or more of Examples 1-3 optionally include wherein the reamer further comprises an extension extending longitudinally between the cutting edge and the handle and removably attached to the cutting edge and the handle, the extension including a plurality of markings, each of the plurality of markings indicative of a cutting depth of the reamer.

In Example 5, the subject matter of any one or more of Examples 1˜4 optionally include wherein the plurality of different implants includes a fracture implant, a limb salvage implant, or a revision implant.

In Example 6, the subject matter of Example 5 optionally includes wherein the plurality of different broach heads includes a fracture implant broach head, a limb salvage implant broach head, and a revision implant broach head.

In Example 7, the subject matter of any one or more of Examples 1-6 optionally include wherein the stem of each of the plurality of different broaches tapers radially towards a central axis as the stem extends longitudinally away from a portion of the stem that attaches to the plurality of different broach heads.

Example 8 is a system for a modular broach and reamer configured to prepare a bone of a patient for a fracture implant, a limb salvage implant, or a revision implant, the system comprising: a plurality of different reamers configured to ream a portion of the bone, each reamer of the plurality of different reamers including: a cutting edge extending longitudinally; and a handle extending longitudinally and removably attached to the cutting edge, the handle configured to rotate the reamer about a central axis of the reamer; and a plurality of different broaches configured to prepare a medullary cavity of the bone to receive an implant, each broach of the plurality of different broaches including: a stem extending longitudinally; and a plurality of different broach heads, each of the plurality of different broach heads configured to prepare the medullary cavity to receive the fracture implant, the limb salvage implant, or the revision implant.

In Example 9, the subject matter of Example 8 optionally includes wherein each reamer of the plurality of different reamers comprises different sizes from one another.

In Example 10, the subject matter of Example 9 optionally includes wherein each broach of the plurality of different broaches comprises different sizes from one another.

In Example 11, the subject matter of Example 10 optionally includes wherein the size of each of the plurality of different reamers is determined by a diameter of the cutting edge, and the size of each of the plurality of different broaches is determined by a diameter of the stem.

In Example 12, the subject matter of any one or more of Examples 8-11 optionally include wherein the cutting edge of each of the plurality of different reamers comprises a plurality of indentations, each of the plurality of indentations indicative of a cutting depth of the reamer.

In Example 13, the subject matter of any one or more of Examples 8-12 optionally include wherein the handle of each of the plurality of different reamers comprises a coupling mechanism, the coupling mechanism engageable with a tool to rotate the plurality of different reamers about the central axis, and wherein the handle also comprises a plurality of markings thereon, each of the plurality of markings indicative of a cutting depth of the reamer.

In Example 14, the subject matter of any one or more of Examples 8-13 optionally include wherein each of the plurality of different reamers comprises an extension extending longitudinally between the cutting edge and the handle and removably attached to the cutting edge and the handle, the extension including a plurality of markings, each of the plurality of markings indicative of a cutting depth of the reamer.

In Example 15, the subject matter of any one or more of Examples 8-14 optionally include wherein the stem of each of the plurality of different broaches tapers radially towards a central axis as the stem extends longitudinally away from a portion of the stem that attaches to the plurality of different broaches.

Example 16 is a method of preparing a bone of a patient to receive an implant by using a modular reamers and broaches, the method comprising: retrieving an implant corresponding to a condition and a size of the bone of the patient; retrieving a reamer corresponding to the size of the retrieved implant; reaming a portion of the bone of the patient; retrieving a broach corresponding to the size and type of the retrieved implant; preparing a medullary cavity of the bone of the patient; and inserting the retrieved implant into the bone of the patient.

In Example 17, the subject matter of Example 16 optionally includes assembling a cutting edge to a handle of the reamer, the cutting edge and handle correspond to the size of the retrieved implant; and assembling a broach head to a stem of the broach, the broach head corresponding to the size and type of the retrieved implant.

In Example 18, the subject matter of Example 17 optionally includes wherein the broach head can be configured to prepare the medullary cavity of the bone of the patient for a fracture implant, a limb salvage implant, or a revision implant.

In Example 19, the subject matter of any one or more of Examples 17-18 optionally include retrieving a second implant, the second retrieved implant a same size and a different style from the retrieved implant; assembling the cutting edge to the handle of the reamer; and assembling a second broach head to the stem of the broach, the second broach head corresponding to the size and type of the second retrieved implant.

In Example 20, the subject matter of any one or more of Examples 16-19 optionally include wherein the size of the retrieved implant is defined by a diameter of a stem of the retrieved implant.

Example 21 is an apparatus comprising means to implement of any of Examples 1-20.

Example 22 is a system to implement of any of Examples 1-20.

Example 23 is a method to implement of any of Examples 1-20.

The above-detailed description includes references to the accompanying drawings, which form a part of the detailed description. The drawings show, by way of illustration, specific embodiments that can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

1. A system of modular reamers and broaches configured to prepare a bone of a patient for one of a plurality of different implants, the system comprising:
a reamer configured to ream a portion of the bone, the reamer including:
a cutting edge extending longitudinally; and
a handle extending longitudinally and removably attached to the cutting edge, the handle configured to rotate the reamer about a central axis of the reamer; and
a broach configured to prepare a medullary cavity of the bone to prepare the bone to receive one of the plurality of different implants, the broach including:
a stem extending longitudinally; and
a plurality of different broach heads, each of the plurality of different broach heads configured to prepare the medullary cavity for one of the plurality of different implants.

2. The system of claim 1, wherein the cutting edge of the reamer comprises a plurality of indentations, each of the plurality of indentations indicative of a cutting depth of the reamer.

3. The system of claim 1, wherein the handle of the reamer comprises a coupling mechanism, the coupling mechanism engageable with a tool to rotate the reamer about the central axis, and wherein the handle of the reamer also comprises a plurality of markings thereon, each of the plurality of markings indicative of a cutting depth of the reamer.

4. The system of claim 1, wherein the reamer further comprises an extension extending longitudinally between the cutting edge and the handle and removably attached to the cutting edge and the handle, the extension including a plurality of markings, each of the plurality of markings indicative of a cutting depth of the reamer.

5. The system of claim 1, wherein the plurality of different implants includes a fracture implant, a limb salvage implant, or a revision implant.

6. The system of claim 5, wherein the plurality of different broach heads includes a fracture implant broach head, a limb salvage implant broach head, and a revision implant broach head.

7. The system of claim 1, wherein the stem of each of the plurality of different broaches tapers radially towards a central axis as the stem extends longitudinally away from a portion of the stem that attaches to the plurality of different broach heads.

8. A system for a modular broach and reamer configured to prepare a bone of a patient for a fracture implant, a limb salvage implant, or a revision implant, the system comprising:
a plurality of different reamers configured to ream a portion of the bone, each reamer of the plurality of different reamers including:
a cutting edge extending longitudinally; and
a handle extending longitudinally and removably attached to the cutting edge, the handle configured to rotate the reamer about a central axis of the reamer; and
a plurality of different broaches configured to prepare a medullary cavity of the bone to receive an implant, each broach of the plurality of different broaches including:
a stem extending longitudinally; and
a plurality of different broach heads, each of the plurality of different broach heads configured to prepare the medullary cavity to receive the fracture implant, the limb salvage implant, or the revision implant.

9. The system of claim 8, wherein each reamer of the plurality of different reamers comprises different sizes from one another.

10. The system of claim 9, wherein each broach of the plurality of different broaches comprises different sizes from one another.

11. The system of claim 10, wherein the size of each of the plurality of different reamers is determined by a diameter of the cutting edge, and the size of each of the plurality of different broaches is determined by a diameter of the stem.

12. The system of claim 8, wherein the cutting edge of each of the plurality of different reamers comprises a plurality of indentations, each of the plurality of indentations indicative of a cutting depth of the reamer.

13. The system of claim 8, wherein the handle of each of the plurality of different reamers comprises a coupling mechanism, the coupling mechanism engageable with a tool to rotate the plurality of different reamers about the central axis, and wherein the handle also comprises a plurality of markings thereon, each of the plurality of markings indicative of a cutting depth of the reamer.

14. The system of claim 8, wherein each of the plurality of different reamers comprises an extension extending longitudinally between the cutting edge and the handle and removably attached to the cutting edge and the handle, the extension including a plurality of markings, each of the plurality of markings indicative of a cutting depth of the reamer.

15. The system of claim 8, wherein the stem of each of the plurality of different broaches tapers radially towards a central axis as the stem extends longitudinally away from a portion of the stem that attaches to the plurality of different broaches.

16. A method of preparing a bone of a patient to receive an implant by using a modular reamers and broaches, the method comprising:
retrieving an implant corresponding to a condition and a size of the bone of the patient;
retrieving a reamer corresponding to the size of the retrieved implant;
reaming a portion of the bone of the patient;
retrieving a broach corresponding to the size and type of the retrieved implant;
preparing a medullary cavity of the bone of the patient; and
inserting the retrieved implant into the bone of the patient.

17. The method of claim 16, further comprising:
assembling a cutting edge to a handle of the reamer, the cutting edge and handle correspond to the size of the retrieved implant; and
assembling a broach head to a stem of the broach, the broach head corresponding to the size and type of the retrieved implant.

18. The method of claim 17, wherein the broach head can be configured to prepare the medullary cavity of the bone of the patient for a fracture implant, a limb salvage implant, or a revision implant.

19. The method of claim 17, further comprising:
retrieving a second implant, the second retrieved implant a same size and a different style from the retrieved implant;
assembling the cutting edge to the handle of the reamer; and
assembling a second broach head to the stem of the broach, the second broach head corresponding to the size and type of the second retrieved implant.

20. The method of claim 16, wherein the size of the retrieved implant is defined by a diameter of a stem of the retrieved implant.
